(19) United States
(12) Patent Application Publication
(10) Pub. No.: US 2006/0271102 A1
(43) Pub. Date: Nov. 30, 2006

(54) Surgical instrument construction

(75) Inventors: Mark R. Bosshard, Shoreview, MN (US); David B. Robinson, Chanhassen, MN (US)

Correspondence Address: NIKOLAI & MERSEREAU, P.A., 900 SECOND AVENUE SOUTH, SUITE 820, MINNEAPOLIS, MN, 55402, US

(73) Assignee: Gyrus Medical, Inc., Maple Grove, MN (US)

(21) Appl. No.: 11/434,376

(22) Filed: May 15, 2006

Related U.S. Application Data
(60) Provisional application No. 60/684,364, filed on May 25, 2005.

(51) Int. Cl. A61B 17/00

(52) U.S. Cl. 606/205

(57) ABSTRACT

A surgical instrument includes a jaw assembly (2) comprising first and second jaw members (3) and (4) pivotally connected one to the other so as to be movable between respective open and closed positions. First and second handle members (8) and (12) are connected to the first and second jaw members respectively, the first and second handle members each including a hollow tubular portion (10) and (14) at the distal end of the handle member. The first and second jaw members (3) and (4) each include an extension (6) and (7) at the proximal end of each jaw member. The external diameter of each extension and the internal diameter of each tubular portion are such that each handle member is connected to a respective one of the jaw members by means of a press-fit connection. To allow for manufacturing tolerances components, the extensions (6) and (7) each include one or more externally facing ribs (19a to 19j), the height of the ribs increasing from a first height towards the first end of the extension to a second height towards the second end of the extension.

### CROSS-REFERENCE TO RELATED APPLICATION

This application claims priority to U.S. Provisional Application Ser. No. 60/684,364, filed May 25, 2005, and which is hereby incorporated by reference.

### BACKGROUND OF THE INVENTION

1. Field of the Invention

This invention relates to a surgical instrument such as a forceps or scissors device, and more particularly, to the manner in which jaw assemblies of such devices are attached to handle members.

2. Discussion of the Prior Art

Such devices have been known for many years, U.S. Pat. No. 2,111,161 dating from 1937 being an early example. The present invention attempts to provide a modern improvement to such devices.

### SUMMARY OF THE INVENTION

Accordingly there is provided a surgical instrument comprising a jaw assembly comprising first and second jaw members pivotally connected one to the other so as to be movable between respective open and closed positions, and first and second handle members connected to the first and second jaw members respectively, the first and second handle members each including a hollow tubular portion at the distal end of the handle member and having a internal diameter, and the first and second jaw members each including an extension at the proximal end of each jaw member having a external diameter such that each handle member is connected to a respective one of the jaw members by means of a press-fit connection.

The instrument is conveniently an open surgical instrument, that is to say an instrument designed to be used in open surgery. Such instruments are similar to the US patent listed above, in that they include a scissors-like handle with apertures into which the surgeon's fingers can be received. This type of instrument is in contrast to those used in endoscopic surgery, which typically have the actuating elements mounted on a long shaft for insertion into a small aperture in the patient's body.

Typically the instrument is a forceps instrument, although it is alternatively conceivably a scissors instrument. The instrument is preferably an electrosurgical instrument, with one or more electrodes mounted on the jaw assembly. The press-fit connection is a permanent connection, as opposed to prior art systems such as U.S. Pat. No. 6,511,480, which provide removable electrode assemblies. The press-fit connection is sufficiently robust to allow for vigorous use of the surgical instrument without the concern that the connection may become detached. Thus the connections of the present invention are “one-time” connections, designed to last for the recommended lifetime of the surgical instrument.

The press-fit connection of the present invention allows for improvements in the cost, weight and component manufacture of the instrument. For example, a single design of handle can be manufactured and connected to different designs of jaw assembly (forceps, scissors etc.) in order to produce a range of instruments. Alternatively, a range of instruments can be provided by means of a single design of jaw assembly, with different designs of handle attached thereto. This may be to accommodate different sizes and shapes for the handles, or merely the preference of one surgeon over another.

According to a preferred arrangement, the hollow tubular portion at the distal end of each handle member is formed of a substantially incompressible material, such as stainless steel. This is in marked contrast to other press-fit connections, which are more generally used with elastomeric materials such as soft plastics and rubber materials. Conveniently, the extension at the proximal end of each jaw member is also formed of a substantially incompressible material, such as stainless steel.

The extension at the proximal end of each jaw member preferably includes a one or more externally facing ribs, the height of each rib increasing from a first height towards the first end of the extension to a second height towards the second end of the extension. For the purposes of this description, the first end of the extension is the end towards the tip of the extension, and the second end of the extension is the end where the extension is connected to the body of the jaw assembly. These terms are used in so that the terms “proximal” and “distal” can be used in an overall sense with regard to the surgical instrument as a whole, as opposed to locally in respect of a particular component such as the extension.

There is preferably a plurality of ribs on the extension, extending either longitudinally along the extension or radially around the extension. The ribs allow for a secure press-fit connection to be established between the jaw assembly and the handles, even when the tubular portion and the extension are subject to manufacturing tolerances. The ribs can accommodate small variations in the sizes of the two components, without producing an unduly loose press-fit connection, or one which is unduly difficult to assemble.

Conveniently, the internal diameter of the hollow tubular portion has a first manufacturing tolerance, and the height of the ribs has a second manufacturing tolerance, the height of the ribs being such that at the Least Material Condition the internal diameter of the hollow tubular potion is between the first height and the second height. The Least Material Condition is herein defined as being the situation in which the extension on the jaw member is the minimum diameter allowed under the manufacturing tolerance, and the internal diameter of the tubular portion is the maximum diameter allowed under the manufacturing tolerance. Thus the Least Material Condition defines the loosest possible fit permissible under the manufacturing tolerances. Even with this loosest fit, the graduation of the heights of the ribs means that one or more of the ribs forms an interference fit with the internal diameter of the tube. Preferably, the height of the ribs is such that at the Least Material Condition the internal diameter of the hollow tubular portion is within the middle third of the range between the first height and the second height. For example, if there are 9 radially extending ribs and a gradual progression in the height of the ribs, the internal diameter of the tubular portion will form an interference fit with ribs in the region of ribs 4 to 6. Thus even at Least Material Condition, the tubular portion will form an interference fit with several of the ribs on the extension.

Additionally, the height of the ribs is preferably such that at the Maximum Material Condition the internal diameter of the hollow tubular portion is greater or equal to the first height. The Maximum Material Condition is herein defined as being the situation in which the extension on the jaw member is the maximum diameter allowed under the manufacturing tolerance, and the internal diameter of the tubular portion is the minimum diameter allowed under the manufacturing tolerance. Thus the Maximum Material Condition defines the tightest possible fit permissible under the manufacturing tolerances. Even with this tightest fit, the graduation of the heights of the ribs means that the internal diameter of the tube fits over at least one of the ribs to form an interference fit.

As stated above, the height of the ribs conveniently increases in a steady progression from the first height to the second height. In an alternative arrangement there can be different groups of ribs, at different heights to other groups of ribs, but with the ribs of each group being all at the same height. In one convenient arrangement there are one or more additional ribs at the first height towards the first end of the extension. This ensures that at the Maximum Material Condition there are several ribs of equal height in interference fit with the tubular member. Alternatively or additionally, there are conveniently one or more additional ribs at the second height towards the second end of the extension.

### DESCRIPTION OF THE DRAWINGS

The invention will now be further described, by way of example only, with reference to the accompanying drawings, in which;

FIG. 1 is a side view of a forceps instrument in accordance with the present invention;

FIG. 1a and 1b are enlarged sectional views of parts of FIG. 1, as shown,

FIG. 2 is a side view of the jaw member component of the instrument of FIG. 1;

FIG. 3 is an enlarged view of a portion of the component of FIG. 2, showing the ribs thereon, and

FIG. 4 is an enlarged view of a portion of the component of FIG. 2 showing an alternative embodiment of the ribs thereon.

### DESCRIPTION OF THE PREFERRED EMBODIMENT

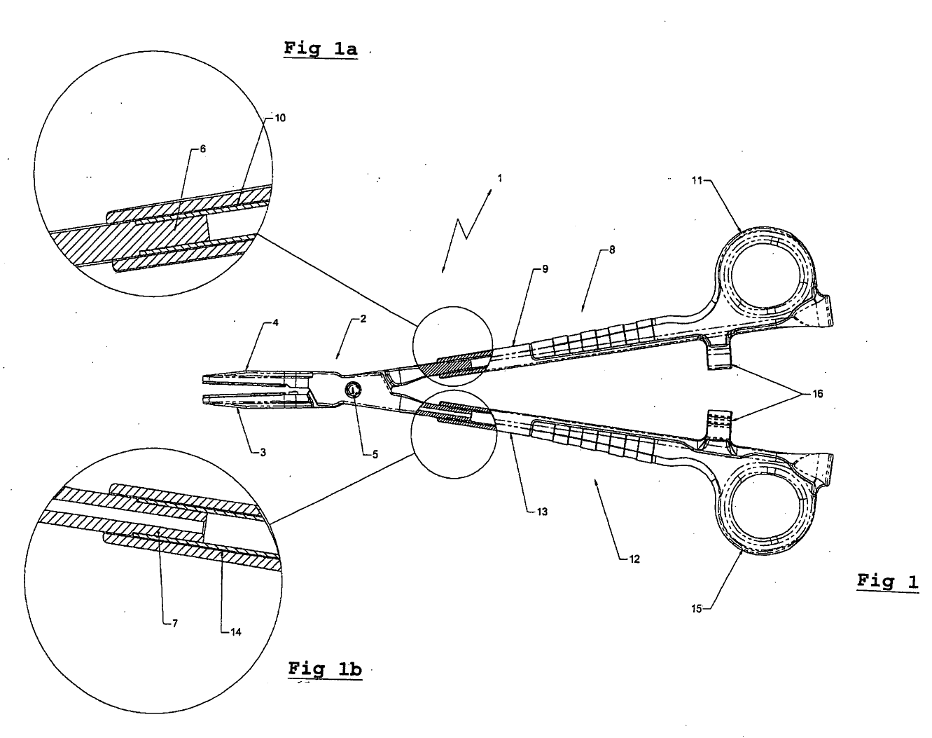

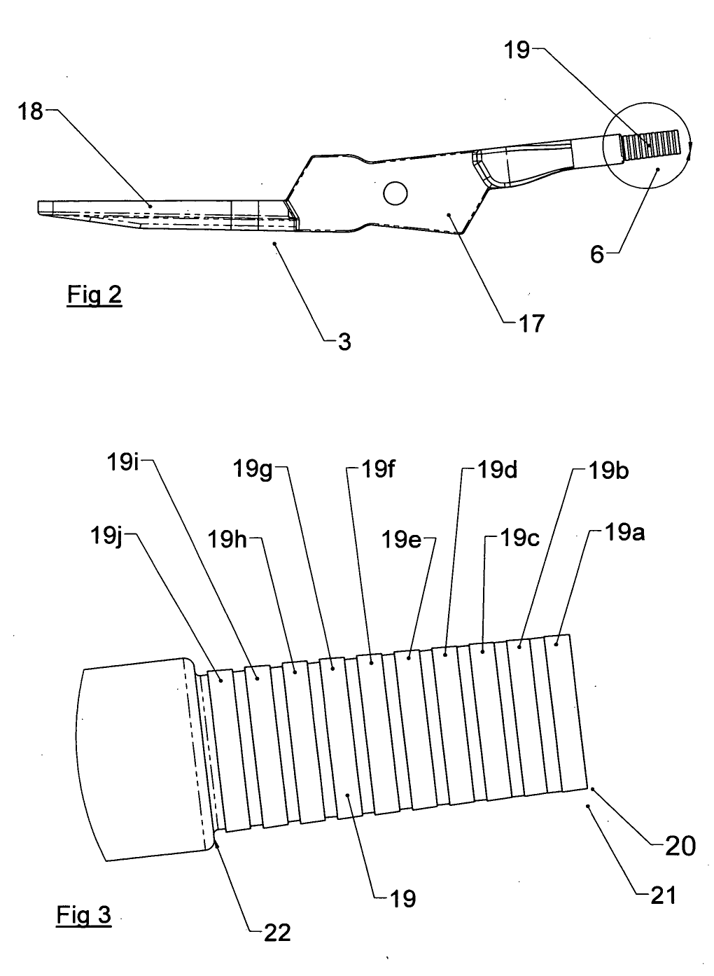

Referring to FIG. 1, an open forceps instrument is shown generally at 1, and comprises a jaw assembly 2 comprising a first jaw member 3 and a second jaw member 4, pivotally connected one to the other by means of pivot pin 5. Jaw member 3 is provided at its proximal end with an extension 6, and jaw member 4 with a similar extension 7.

A first handle member 8 is provided, the handle member comprising a body portion 9, a tubular portion 10 associated with the body portion, and a gripping portion 11 at the proximal end of the handle member. The body portion 9 of the handle member is over-molded over the tubular portion 10. The handle member is attached to the jaw member 3 by means of the tubular portion 10 forming a press-fit connection with the extension 6. In similar fashion, a second handle member 12 is provided, the second handle member also comprising a body portion 13, a tubular portion 14 associated with the body portion, and a gripping portion 15 at the proximal end of the handle member. The handle member is attached to the jaw member 4 by means of the tubular portion 14 forming a press-fit connection with the extension 7.

Movement of the handle members 8 and 12 causes the jaw members 3 and 4 to open and close relative to one another so that tissue can be gripped therebetween. A ratchet mechanism 16 is provided on each handle member for locking the handle members when they are moved together into their closed position.

The jaw member 3 is shown in more detail in FIG. 2. The jaw member comprises a central body portion 17, from which depends the extension 6 in one direction and a jaw element 18 in the other direction. The extension 6 is a solid member having a series of longitudinally-spaced, radial ribs 19 present thereon. These ribs are shown in more detail in FIG. 3.

The extension 6 has 10 ribs 19a to 19j, extending from a tapered lead in portion 20 at a first end 21 of the extension, to the body portion 17 of the jaw member 3 at the second end 22 of the extension. In the example of FIG. 3, the ribs have the following diameters:

| Rib | Diameter |
|---|---|
| 19a | 0.1596 |
| 19b | 0.1596 |
| 19c | 0.1606 |
| 19d | 0.1614 |
| 19e | 0.1624 |
| 19f | 0.1634 |
| 19g | 0.1642 |
| 19h | 0.1652 |
| 19i | 0.1652 |
| 19j | 0.1652 |

All dimensions are in inches and have a tolerance of ±0.001 inches.

The tubular portion 10 of the handle member 8 has an internal diameter of 0.161±0.001 inches. It can therefore vary between 0.0160 and 0.162 inches. At Least Material Condition the tubular portion 10 will have an internal diameter of 0.162, and the ribs will be the values given above minus 0.001. Thus the tubular portion 10 will pass over ribs 19a to 19e and form an interference fit with ribs 19f onwards. Depending on the compressibility of the material (stainless steel) and the force used by the machinery to apply the handle to the jaw member, the tubular portion will be engaged with some or all of ribs 19f to 19j. With automated machinery for applying the handle, the tubular portion can be applied over ribs 19f to 19j without causing the splitting of the tubular portion 10. There will therefore be 5 ribs (19f to 19j) securing the extension 6 within the tubular member 10.

At Maximum Material Condition the tubular portion 10 will have an internal diameter of 0.160, and the ribs will be the values given above plus 0.001. Thus the tubular portion 10 will form an interference fit with all of the ribs, starting from rib 19a onwards. There will therefore be 10 ribs (19a to 19j) securing the extension 6 within the tubular member 10.

As will be seen from the above description, the provision of the ribs 19a to 19j allows for a secure press-fit connection between the handle members (8 and 12) and the jaw members (3 and 4), despite the tolerances permitted for the manufactured components. This can allow the components to be manufactured by molding, with a conventional tolerance of ±0.001 inches, as opposed to being manufactured by precision grinding with a much tighter tolerance.

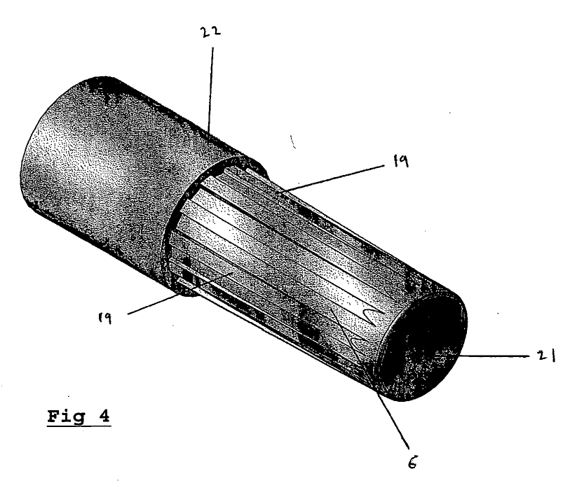

FIG. 4 shows an alternative arrangement in which the ribs 19 extend longitudinally along the extension 6. The ribs slope such that they are at a first relatively shallow height towards the first end 21 of the extension, and a second more pronounced height towards the second end 22 of the extension. The heights of the ribs are such that at the Least Material Condition the internal diameter of the hollow tubular portion 10 is within the middle third of the ribs 19. Thus when the tube 10 is at its loosest, it still engages with a substantial axial length of the longitudinal ribs 19. Similarly, the height of the ribs is such that at the Maximum Material Condition the internal diameter of the hollow tubular portion 10 is greater or equal to the first height. Thus when the tube 10 is at its tightest, it still fits over the shallowest part of the ribs 19 so as to be engaged within their longitudinal extent.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A surgical instrument comprising a jaw assembly comprising first and second jaw members pivotally connected one to the other so as to be movable between respective open and closed positions, and first and second handle members connected to the first and second jaw members respectively, the first and second handle members each including a hollow tubular portion at the distal end of the handle member and having a internal diameter, and the first and second jaw members each including an extension at the proximal end of each jaw member having a external diameter such that each handle member is connected to a respective one of the jaw members by means of a press-fit connection.

2. A surgical instrument according to claim 1 wherein the instrument is an open surgical instrument.

3. A surgical instrument according to claim 1 wherein the instrument is a forceps instrument.

4. A surgical instrument according to claim 1 wherein the instrument is a scissors instrument.

5. A surgical instrument according to claim 1 wherein the instrument is an electrosurgical instrument.

6. A surgical instrument according to claim 1 wherein the hollow tubular portion at the distal end of each handle member is formed of a substantially incompressible material.

7. A surgical instrument according to claim 1 wherein the extension at the proximal end of each jaw member is formed of a substantially incompressible material.

8. A surgical instrument according to claim 1 wherein the extension at the proximal end of each jaw member has a first end towards the tip thereof and a second end towards the body of the jaw assembly, and includes one or more externally facing ribs, the height of the or each rib increasing from a first height towards the first end of the extension to a second height towards the second end of the extension.

9. A surgical instrument according to claim 8 wherein there is provided a plurality of ribs.

10. A surgical instrument according to claim 9 wherein the ribs extend longitudinally along the extension.

11. A surgical instrument according to claim 10 wherein the internal diameter of the hollow tubular portion has a first manufacturing tolerance, and the height of the ribs has a second manufacturing tolerance, the height of the ribs being such that at the Least Material Condition (as hereinbefore defined), the internal diameter of the hollow tubular potion is between the first height and the second height.

12. A surgical instrument according to claim 11 wherein the height of the ribs is such that at the Least Material Condition (as hereinbefore defined), the internal diameter of the hollow tubular portion is within the middle third of the range between the first height and the second height.

13. A surgical instrument according to claim 10 wherein the height of the ribs is such that at the Maximum Material Condition (as hereinbefore defined), the internal diameter of the hollow tubular portion is greater or equal to the first height.

14. A surgical instrument according to claim 10 wherein the height of the ribs increases in a steady progression from the first height to the second height.

15. A surgical instrument according to claim 9 wherein the ribs extend radially around the extension.

16. A surgical instrument according to claim 15 wherein the internal diameter of the hollow tubular portion has a first manufacturing tolerance, and the height of the ribs has a second manufacturing tolerance, the height of the ribs being such that at the Least Material Condition (as hereinbefore defined), the internal diameter of the hollow tubular potion is between the first height and the second height.

17. A surgical instrument according to claim 16 wherein the height of the ribs is such that at the Least Material Condition (as hereinbefore defined), the internal diameter of the hollow tubular portion is within the middle third of the range between the first height and the second height.

18. A surgical instrument according to claim 15 wherein the height of the ribs is such that at the Maximum Material Condition (as hereinbefore defined), the internal diameter of the hollow tubular portion is greater or equal to the first height.

19. A surgical instrument according to claim 15 wherein the height of the ribs increases in a steady progression from the first height to the second height.

20. A surgical instrument according to claim 15 wherein there are one or more additional ribs at the first height towards the first end of the extension.

21. A surgical instrument according to claim 15 wherein there are one or more additional ribs at the second height towards the second end of the extension.
